(19) United States
(12) Patent Application Publication
(10) Pub. No.: US 2026/0207850 A1
(43) Pub. Date: Jul. 23, 2026

(54) PISTON ASSEMBLY AND METHOD FOR OPERATING A PISTON ASSEMBLY

(71) Applicant: LEONARD MIRTSCHIN, BERLIN (DE)

(72) Inventor: STEFFEN MIRTSCHIN, SCHILDOW (DE)

(73) Assignee: LEONARD MIRTSCHIN, BERLIN (DE)

(21) Appl. No.: 19/142,232

(22) PCT Filed: Dec. 20, 2023
(86) PCT No.: PCT/EP2023/087113

§ 371 Date: Jul. 16, 2025

(30) Foreign Application Priority Data
Dec. 23, 2022 (DE) 10 2022 134 740.0

(51) Int. Cl. A61M 5/315; A61M 5/31

(52) U.S. Cl. CPC A61M 5/31513; A61M 5/31528; A61M 2005/3131

(57) ABSTRACT

A piston assembly includes a body and a piston. The body has a first cylindrical cavity. The first cylindrical cavity has an inner wall and a diameter. The piston is shiftably guided in the first cylindrical cavity. The piston has a piston sealing which is arranged at the piston. The piston sealing seals the piston against the inner wall of the first cylindrical cavity and generates sealing forces in a radial direction. The inner wall of the first cylindrical cavity absorbs the sealing forces. The piston sealing is arranged to be stored free from the sealing forces until the piston assembly is used.

### CROSS REFERENCE TO PRIOR APPLICATIONS

This application is a U.S. National Phase application under 35 U.S.C. § 371 of International Application No. PCT/EP2023/087113, filed on Dec. 20, 2023 and which claims benefit to German Patent Application No. 10 2022 134 740.0, filed on Dec. 23, 2022. The International Application was published in German on Jun. 27, 2024 as WO 2024/133566 A1 under PCT Article 21(2).

### FIELD

The present invention relates to a piston assembly containing a body with a first cylindrical cavity and a piston which is shiftably guided in the first cylindrical cavity with a piston sealing for sealing against the inner wall of the first cylindrical cavity, wherein the inner wall of the first cylindrical cavity is configured to absorb sealing forces which are generated in the radial direction by the piston seal. The present invention also relates to a method for operating such a piston assembly.

The present invention also relates to a tribological system with two bodies rubbing against each other in a contact range, wherein at least one of the bodies consists at least in the contact range of a thermoplastic elastomer or another thermoplastic material and a method for reducing friction in such a tribological system.

### BACKGROUND

Typical pistons in the field of syringes and other piston systems in medical technology, such as, for example, for administering medication or another pharmaceutical, operate with a rubber piston which is often coated with silicon in order to reduce the operating power of the devices. There is a risk that unwanted components of the rubber or silicon or the used lubricants dissolve in the liquid with the pharmaceutical. This is particularly disadvantageous in the field of ophthalmology because exogenous substances cannot be broken down in the eye. Such substances accumulate upon repeated application and can cause a negative health impact. Extraneous substances, such as left-over silicon, can negatively affect the effect of the pharmaceuticals.

It is known to use a thermoplastic elastomer as a material for piston assemblies. EP 3222 311 B1 describes a syringe with an uncoated piston-cylinder-system made of thermoplastic elastomer. The use of paraffin as an internal lubricant is thereby described.

The publication “Auswirkungen der Paraffinmigration auf die freie Oberflächenenergie von Naturkautschuk” by A. Ansarifar and Yu Hail in GFKUED 2 63 (2) 69-128 (2010), Dr. Gupta Verlag ISSN 0176-1625 investigates the effect of the migration of paraffin wax on the free surface energy (surface tension) of natural rubber. The surface tension is reduced in an unwanted way with increasing storage duration due to the migration of the wax towards the surface.

The sealings of known piston assemblies with a piston and a piston sealing made of a thermoplastic elastomer will leak overt time. They are therefore not suitable for long storage, such as for two years.

### SUMMARY

An aspect of the present invention is to overcome the disadvantages of the prior art.

In an embodiment, the present invention provides a piston assembly which includes a body and a piston. The body comprises a first cylindrical cavity. The first cylindrical cavity comprises an inner wall and a diameter. The piston is configured to be shiftably guided in the first cylindrical cavity. The piston comprises a piston sealing which is arranged at the piston. The piston sealing is configured to seal the piston against the inner wall of the first cylindrical cavity and to generate sealing forces in a radial direction. The inner wall of the first cylindrical cavity is configured to absorb the sealing forces. The piston sealing is arranged to be stored free from the sealing forces until the piston assembly is used.

### BRIEF DESCRIPTION OF THE DRAWINGS

The present invention is described in greater detail below on the basis of embodiments and of the drawings in which:

FIG. 1 is a schematically illustrated cross section of a piston assembly with a cylindrical cavity and a relief cavity with a piston in a relief position;

FIG. 2 shows the assembly of FIG. 1 with the piston in a sealing position;

FIG. 3 is a longitudinal cross section of the piston rod with a latching mechanism according to a first embodiment of the present invention;

FIG. 4 is a perspective illustration of the latching mechanism of FIG. 3 in greater detail;

FIG. 5 is a cross section of the piston rod along the section line shown in FIG. 3;

FIG. 6 is a top view on the piston rod in FIG. 3;

FIG. 7 is a separate illustration of a clamp for the latching mechanism in FIG. 3;

FIG. 8 is a longitudinal cross section of the clamp in FIG. 3;

FIG. 9 is a longitudinal cross section of the piston rod of FIG. 3 in a syringe housing where the piston sealing is stored in a relief cavity;

FIG. 10 shows the piston sealing of FIG. 9 in greater detail;

FIG. 11 shows the assembly of FIG. 9 where the piston is sealingly guided with the piston sealing in the cylindrical cavity of the syringe housing;

FIG. 12 shows the piston sealing of FIG. 11 in greater detail;

FIG. 13 is a longitudinal cross section of a piston assembly in delivery condition according to a second embodiment of the present invention;

FIG. 14 is a detail of FIG. 13;

FIG. 15 is a longitudinal cross section of the piston assembly of FIG. 13 in a relief position;

FIG. 16 is a detail of FIG. 13;

FIG. 17 shows the assembly of FIG. 13 where the piston is sealingly guided with the piston sealing in the cylindrical cavity of the housing;

FIG. 18 is a detail of FIG. 17;

FIG. 19 shows a piston with a separated piston rod according to a third embodiment of the present invention; and

FIG. 20 shows the piston of FIG. 19 with connected piston rod.

### DETAILED DESCRIPTION

The present invention provides an assembly which is characterized in that the piston sealing positioned at the piston is storable free from sealing forces until use. A relief cavity can, for example, be provided in which the piston sealing positioned at the piston is storable free from sealing forces until use.

The present invention is based on the discovery that the thermoplastic material of the sealing is exposed to radial sealing forces in the piston which cause a pressure deformation of the material and thereby a reduction of the diameter of the sealing. The remains of the pressure deformation causes leaks over time.

The present invention provides that the piston sealing present at the piston is stored free from sealing forces until use. A relief cavity is provided therefor. The relief cavity has the effect that the piston can be stored with the sealing under sterile conditions outside of the first cylindrical cavity for an extended period without being exposed to forces. No pressure deformation exists. The sealing will therefore have the same diameters even after an extended storage. The sealing power is not negatively affected by the storage. The regulations relating to the expiration date of, for example, two or more years can thereby in particular be reliably fulfilled.

The present invention provides an assembly with a long minimum durability, a good sealing, a high even sliding capability, and a considerably reduced stick-slip-effect without the risk of dissolving unwanted substances into the administered liquid.

Only with inserting the piston sealing into the first cylindrical cavity will radial sealing forces be exerted. Since this is performed only shortly prior to use, such as, for example, some minutes before, the pressure deformation during this time can be neglected.

The present invention enables the use of materials with a high pressure deformation remains, such as a thermoplastic elastomer, thermoplastic urethane, and materials from the group of liquid silicon rubber (LSR).

The piston sealing can be stored in a separate relief cavity alone or together with further piston sealings. The piston sealing must then be removed with or without the piston attached thereto from the relief cavity and transferred into the first cylindrical cavity. This involves a risk of contamination. It can, for example, be provided that the relief cavity be connected to the first cylindrical cavity, forms a second cylindrical cavity extending coaxially with the first cylindrical cavity, and has a diameter which is larger than the diameter of the first cylindrical cavity. A sealing already attached to the piston can be stored in the second cylindrical cavity with this embodiment of the present invention. No radial sealing forces are exerted on the sealing in such a second cylindrical cavity. The second cylindrical cavity may be sterile. The piston can be moved in an axial direction without contact to the environment directly from the second cylindrical cavity into the first cylindrical cavity. Any risk of contamination is thereby avoided.

In an alternative modification, the present invention provides that the piston is configured to be connected to a profiled piston rod coaxially extending through the first and the second cylindrical cavity, wherein the piston is provided with a profiled cavity engaging with the profiled piston rod upon establishing the connection and widening the piston so that radial sealing forces are generated.

A piston rod is useful in particular with simple and inexpensive syringes. The piston is moved by pressure on the end of the piston rod which is remote to the exit of the syringe or by pulling in the opposite direction. The piston rod in this embodiment is additionally used to widen the piston and to establish sealing forces. No additional relief cavity is thereby necessary. The cylinder can have the same diameter along its entire length.

A releasable locking mechanism can, for example, be provided for locking the piston sealing in its position in the relief cavity until use. It is thereby avoided, for example, that the piston sealing accidently enters the first cylindrical cavity during transport and is prematurely exposed to radial sealing forces.

In a further modification, the present invention provides that:
(a) the cylindrical cavity is enclosed by a housing;
(b) the locking mechanism comprises a clamp with an elastic recess guiding the piston rod; and
(c) the locking mechanism comprises a bulge at the piston rod forming a resistance which can be overcome only by exerting an additional force upon passing the elastic recess when the piston sealing is moved into the first cylindrical cavity.

The lock can be provided via a wedge or a clamping device.

The bulge at the piston rod can in particular be provided in a range which will not enter the first cylindrical cavity at any time before contact with the piston. This avoids contamination and the insertion of particles into the first cylindrical cavity.

The movement of the piston into the first cylindrical cavity is influenced with the locking mechanism. A further bulge may additionally be provided at the piston rod for limiting the movement of the piston in the direction out of the first cylindrical cavity. It is thereby avoided that the piston is accidently removed from the sensitive sealed space in the first cylindrical cavity or the relief cavity. A limitation can, for example, be provided which avoids the piston sealing moving back into the relief cavity. It is thereby avoided in a syringe or a PRP kit, for example, that medical fluid is drawn up into the relief cavity and causes the device to leak.

An embodiment of the present invention provides that the bulge is formed by a ball held in the piston rod, the ball having a diameter which is selected so that it extends beyond the edge of the piston rod. The ball can rotate when passing the recess of the clamp, whereby no or only minimum abrasion occurs. The bulge is alternatively formed by integrated projections.

In the assembly according to the present invention, the piston may comprise a head made of rubber, thermoplastic elastomer, or any other suitable plastic material which is attached on the piston rod, the head having at least one integrated coaxial circumferential ring forming the piston sealing. The piston rod can be made of quasi any, in particular of any stiff material. It is important that the head and the sealing in medical uses consists of materials which do not cause any dissolving of undesired substances into the liquid present in the cylindrical cavity.

The piston assembly is suitable for a great many different uses. Particularly advantageous is, however, the use of the piston assembly to form a portion of a syringe or a PRP kit.

In an embodiment, the present invention provides that the piston or the piston rod is provided with a threaded spindle which cooperates with a thread fixed within the housing, whereby a piston movement can be effected via a rotational movement. Such an embodiment is particularly useful if the volume of the cylindrical cavity, as limited by the piston, is to be finely adjusted. This is, for example, the case if a certain fraction of the liquid is to assume a certain range of the cavity. The piston can then be moved into the position with high precision where the level of this fraction is at the desired height.

In a further modification of the present invention, a latching mechanism or any other mechanism is provided which locks upon inserting the piston sealing at the piston into the first cylindrical cavity or prevents in another way that the piston sealing moves out of the first cylindrical cavity. The latching mechanism prevents the piston from again being removed and thereby contaminated outside of the cylindrical cavity.

A method for operating a piston assembly according to the present invention is characterized in that the piston with the piston sealing is stored free from sealing forces until use outside of or inside the first cylindrical cavity. No pressure deformation therefore occurs during storage. Contamination by unwanted substances in the environment or faulty handling is avoided if the piston with the piston sealing is stored in a relief cavity which is connected to the first cylindrical cavity.

The present invention also provides a tribological system with two bodies rubbing against each other in a contact range, wherein at least one of the bodies consists at least in the contact range of a thermoplastic elastomer or another thermoplastic material characterized in that the thermoplastic material has a paraffin content and is stored in the air or in another oxygen comprising gas, whereby paraffin moves to the surface. Contrary to the above-mentioned publication by Ansarifar et al., the effect of moving paraffin is desired in some tribological systems. In a first variation, the system is stored until sufficient paraffin is moved to the surface. Such a system is, for example, a system, where the body is a piston with a piston sealing of thermoplastic elastomer having a paraffin content. The minimum storage duration results from the selection of the material, the paraffin content, and the desired sliding capability of the tribological system. A high paraffin content and a long storage duration will result in a good sliding capability.

It has been found that a good sliding capability can already be achieved if the thermoplastic material is stored for at least 8 days, for example, for at least 10 days.

The body may in particular be a piston with a piston sealing which is made of a thermoplastic elastomer having a paraffin content. The piston can be guided in a cylindrical cavity.

A particularly advantageous method of the present invention provides that a body which consists at least in the contact range of a thermoplastic elastomer or any other thermoplastic material and which comprises a paraffin content is stored in the air or in an oxygen containing gas before use, whereby paraffin moves to the surface.

In order to reduce the required operating forces for the piston movement, the contact range of the body may be coated with paraffin before use, wherein the coating of the contact range can be effected with a second body which also consists at least partially of a thermoplastic elastomer having a paraffin content. A piston assembly, for example, a separately stored gliding body, can thereby be used for the coating of the inner wall with paraffin during manufacturing.

The body can be a piston with a piston sealing or any other freely formed piece with a rounded geometry which is made of a thermoplastic elastomer having a paraffin content. The body may then be guided in a cylindrical cavity, the inner wall being coated by the sliding movement with paraffin before the final piston is used.

In this description and in the appended claims, all terms have the meaning which is well known to the person skilled in the art which is defined in technical literature, norms, and in relevant internet sites and publications, in particular of the lexical kind, such as www.wikipedia.de, www.wissen.de or of competitors, research institutes, universities and associations. The terms here used do not have the opposite meaning of what the person skilled in the art will derive from the above publications.

The present invention is described in greater detail below based on embodiments as shown in the drawings.

### First Embodiment: Syringe

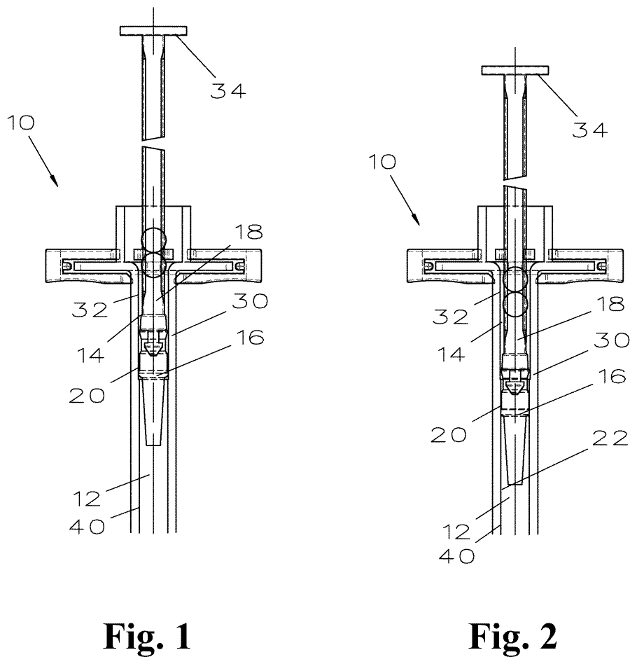

FIGS. 1 and 2 schematically show a piston assembly which is generally designated by numeral 10 with a housing 30. The housing 30 has a section 40 with a cylindrical cavity 12 and a section 32 with a coaxial, also cylindrical relief cavity 14.

FIG. 1 shows a piston 16 present in the relief cavity 14 which is connected to a piston rod 18. The piston 16 is provided with an all-round piston sealing 20. The piston 16 in FIG. 1 is in a position where the piston sealing 20 is positioned inside the relief cavity. The cross-sectional diameter of the relief cavity 14 is selected so that no radial sealing forces are exerted on the piston sealing 20 and the piston 16 as shown in FIG. 1. There is no pressure deformation of the piston sealing 20 in this position. The piston assembly 10 can be stored long-term with the piston sealing in the relief cavity.

With pressure on the end 34 of the piston rod 18, the piston 16 can be moved downwards in the representation for the use of the piston assembly 10. FIG. 2 shows the piston 16 in a position where the piston sealing 20 is positioned in the cylindrical cavity 12. In this position, radial sealing forces are exerted by the inner wall 22 of the cylindrical cavity 12 on the piston sealing 20. The range 24 before the piston 16 is sealingly separated from the remaining range inside the housing 30.

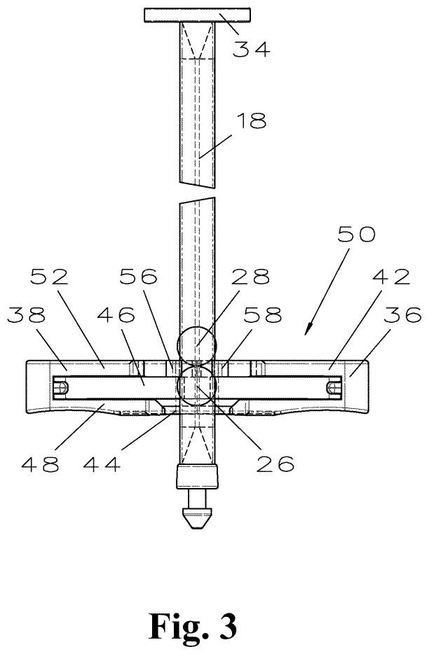

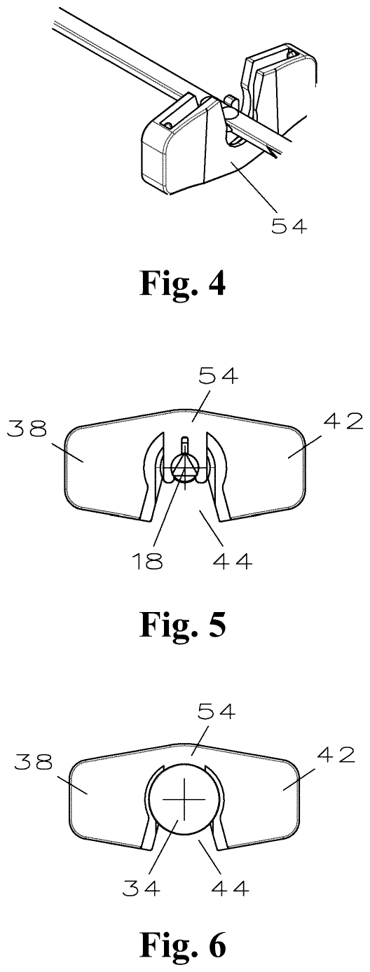

The piston assembly 10 can, for example, be part of a syringe and be used for applying pharmaceuticals. It is then important that the piston 16 and the sections of the piston rod 18 which enter the cavities 12 and 14 are stored fully sterile until use. A particularly simple handling is achieved if the piston 16 is already inserted into the housing 30 by the manufacturer and is transported and stored in such a position. For use, one must only exert pressure on the end 34 in the direction of the syringe opening. It is thereby important that the piston 16 cannot slip or be pulled out of the relief cavity 14 prior to use. In order to avoid the removal of the piston 16 from the housing 30, a latching mechanism is provided, the set-up and function of which is illustrated in FIGS. 3 to 6.

FIG. 3 is a longitudinal cross section of the piston rod 18 and the latching mechanism 50. The piston rod 18 has a triangular cross section in the present embodiment. This can be recognized in FIG. 5. The latching mechanism 50 comprises two balls 26 and 28 which are positioned inside the piston rod. This can be recognized in FIG. 3. The ball 28 remote to the piston 16 is thereby slightly larger than the other ball 26. Both balls 26 and 28 have a diameter extending through openings beyond the plane sides of the piston rod 18. Balls 26 and 28 thereby form a bulge at the piston rod 18. It is understood that an integrated bulge may also be used.

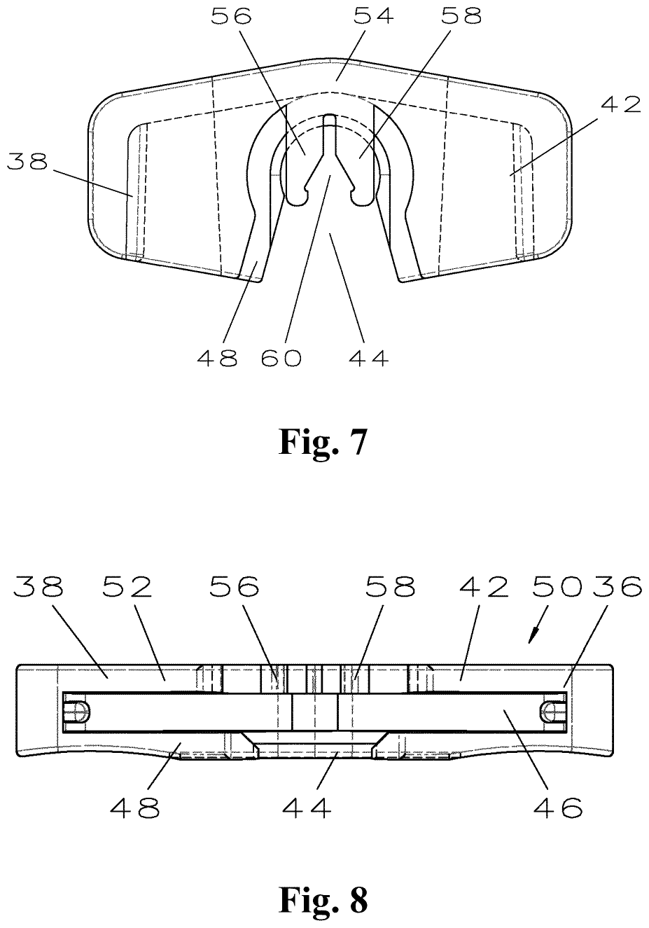

The latching mechanism 50 of the present embodiment also comprises a clamp 36 which is separately shown in FIGS. 7 and 8. The clamp 36 comprises two flat double layer wings 38 and 42. The wings 38 and 42 are shown to extend in a horizontal direction. The wings 38 and 42 are arranged opposite each other around a recess 44 which is open at the longitudinal side. A gap 46 is provided between the layers 48 and 52 of the wings 38 and 42 which extends up to an edge along the entire length of the wings 38 and 42. This can be recognized in FIG. 8. In the plane of the layer 52 facing the end 34 (at the top in FIG. 8), the recess 44 is slightly larger than in the plane of the in FIG. 8 lower layer 48. This is shown in FIGS. 3 and 7.

The recess 44 extends from an open side (at the lower end in FIG. 5) to a bridge 54 connecting the wings 38 and 42. Two tongues 56 and 58 are connected to the bridge 54 in the plane of the layer 52 (at the upper end in FIGS. 3 and 8) as shown in FIG. 7. The tongues 56 and 58 are sloped at the longitudinal sides facing each other, whereby a triangular receptacle 60 is formed. The free ends project slightly inwardly and are rounded.

The clamp 36 can be form fittingly clipped onto the piston rod 18 with the receptacle 60 through a slit in the housing 30, which is horizontal in the representation as shown in FIG. 7. The plane of the layer 52 will then be positioned between the balls 26 and 28. The projecting free ends of the tongues 56 and 58 form a resistance and hold the clamp 36 in its lateral position. No shifting or rotation of the clamp in the plane of the layer 52 is therefore possible. The balls 26 and 28 form a resistance for the tongues 56 and 58 and secure the axial position of the clamp 50 at the piston rod 18.

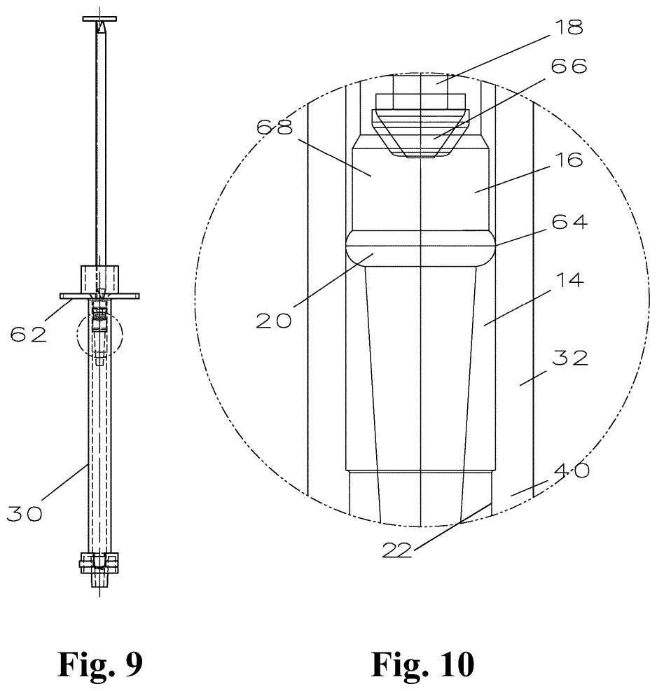

In the range before the end of the housing 30 remote to the needle, two opposite wings 62 are connected to the housing 30 in the usual way as shown in FIG. 9. The wings 62 serve as an abutment when exerting pressure on the end 34 of the piston rod 18. In the present embodiment, the clamp 36 has a size where it can be shifted onto the wings 62 of the housing 30 with a gap 46 between the layers 48 and 52. The piston rod 18 and the piston 16 fixed thereto are secured against movement in an axial direction.

The balls 26 and 28 have a position in the piston rod 18, where the piston sealing 20 at the piston 16 is accommodated in the relief cavity 14, when the piston rod 18 is locked with the locking mechanism 50 as described above. The piston assembly 10 can be stored and transported well in this condition. The locking of the piston in such a storage position is effected from the outside, whereby contamination by abrasion or other unwanted particles is avoided. No pressure is exerted on the piston sealing 20.

The locking mechanism 50 must be released to use the syringe. Pressure is exerted on the end 34 of the piston rod for this purpose. The tongues 56 and 58 can be slightly moved apart upon sufficient pressure in a lateral direction. The piston rod 18 is pushed with the bulge in the form of the in FIG. 3 lower ball 26 through the receptacle 60. The piston sealing 20 is thereby moved into the cylindrical cavity 12 with the smaller diameter. The pharmaceutical can then be pulled up from a vial and be injected or a pharmaceutical already present in the syringe can be injected. The slightly larger ball 28 prevents the piston 16 from moving back into the storage and transport position and from too much liquid being pulled up and to enter the relief cavity.

In the present embodiment, an assembly is used with a piston rod 18 and a separate piston 16 in the form of a head with integrated piston sealing 20 made of a thermoplastic elastomer. It is understood that the assembly may also be uniformly made of one piece from a stiff material with a sealing groove for a separate sealing.

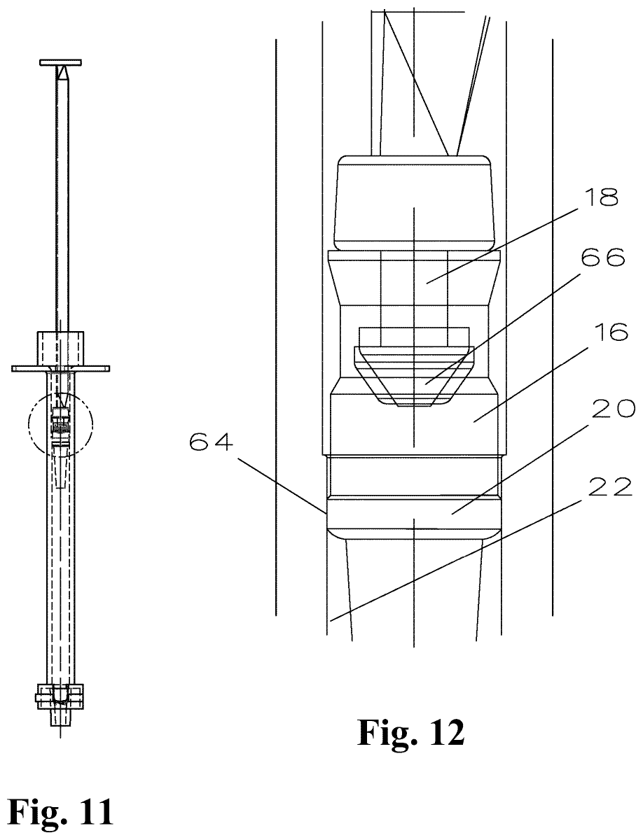

FIGS. 9 and 12 show the assembly in greater detail. FIG. 9 shows the entire assembly with a circumferential, annular piston sealing 20 in the relief cavity 14. FIG. 11 shows the assembly of FIG. 9 after the activation of the use with the piston sealing 20 in the cylindrical cavity 12. when comparing the detailed illustrations in FIGS. 8 and 10, it can be recognized that in this position a pressure exists between the piston sealing 20 and the inner wall 22 which causes a slight deformation of the piston sealing 20 in the range 64. The rounded form of the piston sealing 20 shown in FIG. 10 is pressed against the inner wall 22 when in the position in FIG. 12 and seals the range 64 before the piston 16 well.

The elastic material of the piston 16 with the piston sealing 20 forms a head which is conically reduced on the side of the piston sealing 20 remote to the piston rod 18. An easy introduction into the smaller portion 40 of the housing 30 is thereby enabled. The portion 68 on the other side of the piston sealing 20 is provided with a recess at its end face. The elastic material is stuck onto the funnel-shaped end 66 of the piston rod with this recess. The recess is slightly smaller than the funnel-shaped end 66 of the piston rod 18, whereby the material is slightly widened and under tension. The piston 16 cannot therefore be removed from the piston rod 18.

### Second Embodiment: Separation of a Liquid Fraction

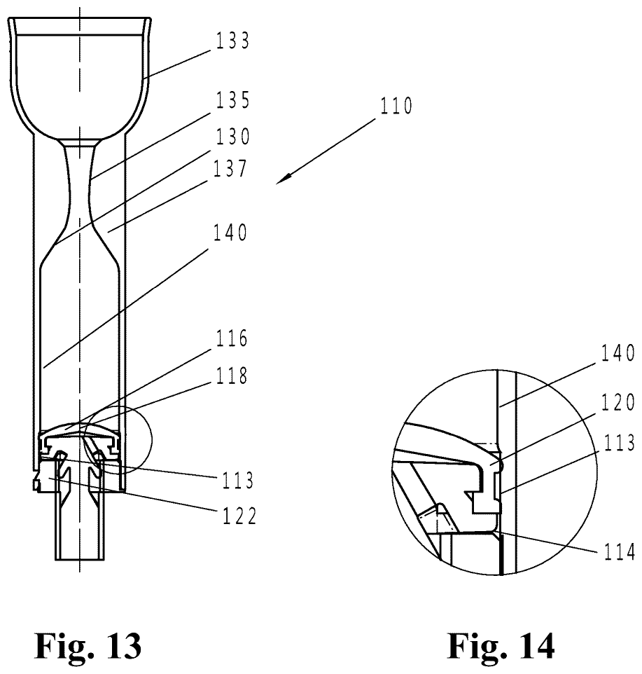

The second embodiment shown in FIGS. 13 to 18 relates to a piston assembly 110 with a piston 116, the advancement of which is achieved by rotation. The piston assembly 110 comprises a housing 130 and the piston 116. The housing 130 has an upper housing section 133, a lower housing section 140, and a middle housing section 135 in-between, which is formed by a taper with a small cross-sectional diameter. The middle housing section 135 is reinforced with four reinforcing ribs 137. The reinforcing ribs 137 extend in a star shape in the vertical direction in the representation around the middle housing section 135 and are connected to all housing sections. The reinforcing ribs 137 stabilize the piston assembly 110.

A liquid introduced into the housing 130 closed with a lid, for example blood, can be centrifuged or divided into fractions in any other way in the housing 130. The volumes in the housing sections are selected so that a selected fraction of the liquid is located in the middle housing section 135. It may then be easily removed using, for example, a pipette.

The fluid levels of the fractions vary depending on the composition of the fluid. The level of the DNA and long-chain protein-containing fraction in blood, for example, depends on the physiology of the person from whom the blood was taken. The volume in the lower housing section 140, and thus the level of the fraction in the housing section 135, can be adjusted using the piston 116.

Since the piston 116 serves to finely adjust the volume in the lower housing section 140, the adjustment in the present embodiment is not made by applying pressure to the piston. The piston 116 is rather connected to a threaded spindle 118. The threaded spindle 118 is screwed into a thread 122 that is fixed to the housing 130. The thread 122 can, for example, be part of a plug which is inserted from below into an opening in the housing 130. By rotating the threaded spindle 118, for example, via a rotary handle that can be inserted into the threaded insert with the thread 122, the piston 116 is moved in an axial direction. The volume in the lower housing section 140 is thereby changed and the level of the overlying fractions can be adjusted to a desired level.

As in the first embodiment, the piston 116 is provided with a head which is made of a thermoplastic elastomer. A piston sealing 120 is molded to the head. Before the assembly is used, the head is located in a relief cavity 114 in a housing section 113 below the lower housing section 140. As in the first embodiment, the relief cavity 114 has a larger diameter than the connected coaxial cavity in the lower housing section 113.

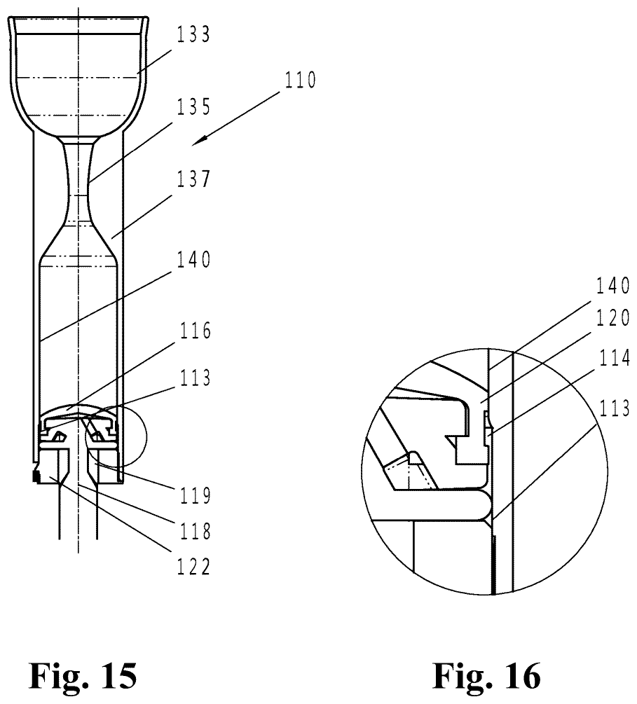

FIG. 14 shows the storage and transport state of the piston assembly 110 from FIG. 13 in greater detail. It can be recognized that the piston sealing 120 is not exposed to any radial forces and accordingly no compression set occurs. FIG. 16 shows the piston assembly 110 of FIG. 15 in greater detail. The piston sealing 120 is located just above the annular shoulder, which is formed between the lower housing section 140 and the housing section 113 with the relief cavity 114. Sealing forces in this state are exerted on the piston sealing 120.

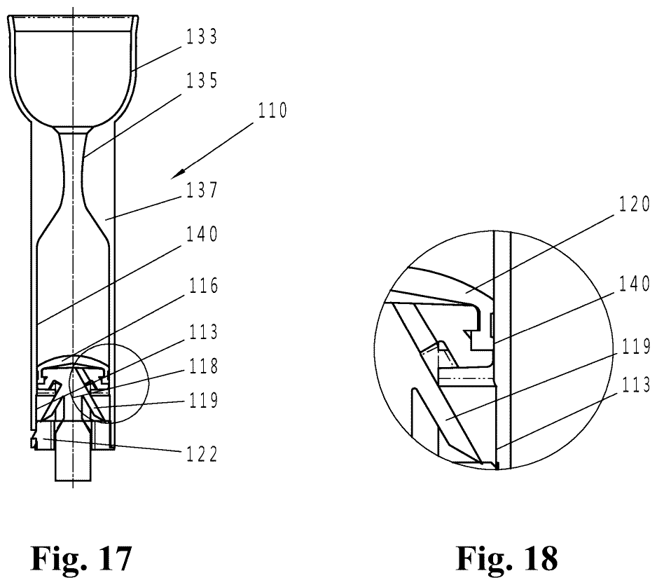

A latching mechanism is provided in order to prevent the piston 116 from being rotated back and the piston sealing 120, once it has entered the range of the narrower housing section 140, from returning to the relief cavity 114. The latching mechanism comprises two opposing latching elements 119 which are formed on the threaded spindle 118. In the storage and transport state, which is shown in FIGS. 13 and 14, the latching elements 119 are in close contact and are held in position by the threaded insert with thread 122. When the piston 116 is moved upwards, the lower edge of the latching elements 119 is also moved out of the threaded insert with thread 122. The latching elements 119 are kept under tension. If the latching elements 119 are no longer held in position by the threaded insert (FIGS. 11 and 12), they snap outwards. This is illustrated in FIGS. 17 and 18. They will then form a resistance that can no longer be overcome without destruction, which prevents the piston 116 from moving back downwards. It is understood that any other locking or latching mechanism suitable for moving the piston 116 back can also be used.

The piston can be made of a thermoplastic, for example TPE (SEBS basis) thermoplastic elastomer with modification parafinole, TPO/TPE-O thermoplastic polyolefin elastomers, TPU thermoplastic polyurethane with modification parafinole or PP/PE with parafinole modification (particularly soft settings) or of a thermoset, for example, LSR liquid silicone rubber with modification parafinole. The piston is here made of TPE.

A thermoplastic can be chosen for the cylinder, for example, PETG polyethylene terephthalate glycol-modified, PET polyethylene terephthalate, PBT polybutyl terephthalate, COC cyclo olefin copolymer or PMMA polymethyl methacrylate. A cylinder made of PETG was chosen for the present embodiment.

### Third Embodiment: Separate Piston Rod

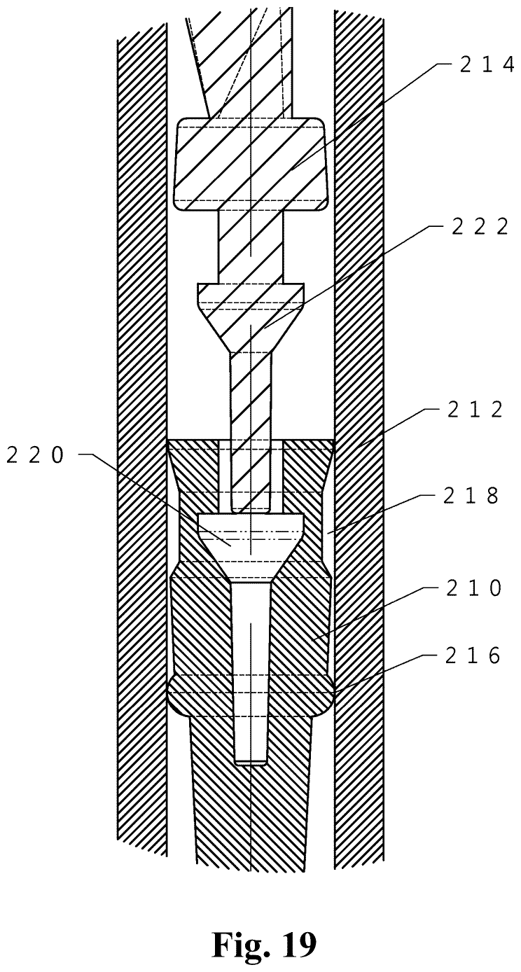

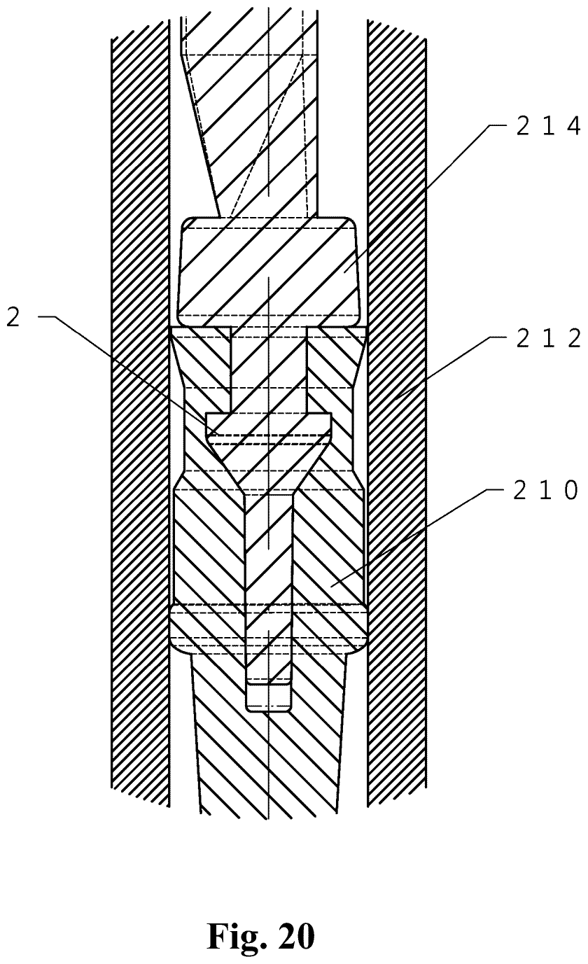

A third embodiment is shown in FIGS. 19 and 20. The piston 210 is inserted into a cylinder 212 with a continuous inner diameter. During storage, the piston 210 is separated from the piston rod 214. There is no connection between the piston 210 and the piston rod 214 and their inner filling contour. The piston 210 in this assembly is stored securely and largely free of the radial forces and associated compression set. This can be seen in FIG. 19. The inner diameter of the cylinder 212 just corresponds to the diameter of the piston 210 or, in some cases, its later slight oversize relative to the sealing function at the point of the largest diameter 216. A gap 218 is otherwise formed between the inner wall of the cylinder 212 and the piston 210. The soft, elastic piston 210 can yield and deform without changing its dimensions.

Joining of the piston rod 214 with connection into the piston 210 is provided only shortly prior to use. The connection is created by an irreversible click by pushing the fixed piston rod 214 in the direction of the elastomer piston 210. A positive fit is created which enables movement in the longitudinal direction. This is illustrated in FIG. 20. It can here be recognized that the piston 210 has an opening on the side facing the piston rod 214 which opens into a profiled cavity 220. The profile of the cavity 220 corresponds approximately to the outer profile 222 of the piston rod 214.

FIG. 19 shows that the elastic piston can yield to external pressure by deformation. FIG. 20 shows the assembled piston with piston rod 214. The lower part of the outer profile 222 of the piston rod 214 sits firmly in the cavity 220. The piston 210 is pressed slightly outwards, similar to a dowel, and seals completely.

This design enables storage at different temperatures, for example, sterilization with EO gas at over 50° C. and over a period of, for example, 24 hours.

The exemplary embodiments explained above serve to illustrate the present invention claimed in the claims. Features that are disclosed together with other features can generally also be used alone or in combination with other features that are explicitly or implicitly disclosed in the text or drawings in the exemplary embodiments. Dimensions and sizes are given as examples only. Suitable ranges can be derived by the knowledge of the person skilled in the art and therefore do not need to be explained here in greater detail. The disclosure of a specific embodiment of a feature does not mean that the present invention should be limited to said specific embodiment. Such a feature can rather be implemented using a variety of other configurations that are familiar to those skilled in the art. The present invention can therefore be embodied not only in the form of the embodiments explained, but also in all embodiments which are covered by the scope of the appended claims.

The terms “top”, “bottom”, “right” and “left” exclusively refer to the attached drawings. It is understood that claimed devices also can adopt a different orientation. The term “including” and the term “comprising” mean that further components not mentioned may be provided. The terms “essentially” and “predominantly” include all characteristics that have a majority of a property or content, i.e., more than all the other components or properties of the feature mentioned, i.e., in the case of two components, for example, more than 50%.

The present invention is not limited to embodiments described herein; reference should be had to the appended claims.

### List of Reference Numerals

10 Piston assembly
12 Cylindrical cavity
14 Cylindrical relief cavity
16 Piston
18 Piston rod
20 Piston sealing
22 Inner wall
26 Ball
28 Ball
30 Housing
32 Section (of housing 30 with relief cavity 14)
34 End (of piston rod 18)
36 Clamp
38 Wing
40 Section (of housing 30 with a cylindrical cavity 12)
42 Wing
44 Recess
46 Gap
48 Layer
50 Latching mechanism
52 Layer
54 Bridge
56 Tongue
58 Tongue
60 Receptacle
62 Wing
64 Range
66 Funnel-shaped end
68 Portion (on other side of piston sealing 20)
110 Piston assembly
113 Housing section
114 Relief cavity
116 Piston
118 Threaded spindle
119 Latching elements
120 Piston sealing
122 Thread
130 Housing
133 Upper housing section
135 Middle housing section
137 Reinforcing ribs
140 Lower housing section
210 Piston
212 Cylinder
214 Piston rod
216 Largest diameter
218 Gap
220 Cavity
222 Outer profile (of piston rod 214)

What is claimed is:

1-20. (canceled)

21. A piston assembly comprising:
a body comprising a first cylindrical cavity, the first cylindrical cavity comprising an inner wall and a diameter; and
a piston which is configured to be shiftably guided in the first cylindrical cavity, the piston comprising a piston sealing which is arranged at the piston, the piston sealing being configured to seal the piston against the inner wall of the first cylindrical cavity and to generate sealing forces in a radial direction,
wherein,
the inner wall of the first cylindrical cavity is configured to absorb the sealing forces, and
the piston sealing is arranged to be stored free from the sealing forces until the piston assembly is used.

22. The piston assembly as recited in claim 21, further comprising:
a cylindrical relief cavity comprising a diameter, the cylindrical relief cavity being configured to store the piston sealing free from the sealing forces until the piston assembly is used.

23. The piston assembly as recited in claim 21, wherein,
the cylindrical relief cavity is connected to the first cylindrical cavity,
the cylindrical relief cavity forms a second cylindrical cavity which is arranged to extend coaxially with the first cylindrical cavity, and
the diameter of the cylindrical relief cavity is larger than the diameter of the first cylindrical cavity.

24. The piston assembly as recited in claim 23, further comprising:
a profiled piston rod comprising a longitudinal axis, the profiled piston rod being arranged to extend through the first cylindrical cavity and through the second cylindrical cavity,
wherein,
the piston is further configured to be coaxially connected to the profiled piston rod,
the piston further comprises a profiled cavity which is configured to engage with the profiled piston rod upon an establishment of a connection with the profiled piston rod, and
the profiled cavity is arranged to widen the piston so as to generate the radial sealing forces.

25. The piston assembly as recited in claim 24, further comprising:
a housing comprising a thread fixed therein,
wherein,
the piston further comprises or the profiled piston rod further comprises a threaded spindle,
the threaded spindle is configured to cooperate with the thread fixed within the housing, and
the piston is configured to move axially via a rotational movement of the piston or of the profiled piston rod.

26. The piston assembly as recited in claim 23, further comprising:
a releasable locking mechanism which is configured to lock the piston sealing in a position in the cylindrical relief cavity until the piston assembly is used.

27. The piston assembly as recited in claim 26, further comprising:
a housing,
wherein,
the first cylindrical cavity is enclosed by the housing,
the releasable locking mechanism comprises a clamp, the clamp comprising an elastic recess which is configured to guide the profiled piston rod, and
the releasable locking mechanism further comprises a bulge at the profiled piston rod, the bulge being configured to form a resistance which can be overcome only by exerting an additional force upon passing the elastic recess when the piston sealing is moved into the first cylindrical cavity.

28. The piston assembly as recited in claim 27, further comprising:
a further bulge at the profiled piston rod, the further bulge being configured to limit a movement of the piston in a direction out of the first cylindrical cavity.

29. The piston assembly as recited in claim 28, wherein,
the bulge is formed by a ball which is held in the profiled piston rod, and
a diameter of the ball is selected so as to extend beyond an edge of the profiled piston rod.

30. The piston assembly as recited in claim 26, wherein,
the piston further comprises a head which is made of a rubber, a thermoplastic elastomer or of a plastic material,
the head is attached on the profiled piston rod, and
the head comprises at least one integrated coaxial circumferential ring which forms the piston sealing.

31. The piston assembly as recited in claim 21, wherein the piston assembly forms a portion of a syringe or of a PRP kit.

32. The piston assembly as recited in claim 21, further comprising:
a latching mechanism which is configured to lock upon an insertion of the piston sealing into the first cylindrical cavity or which otherwise prevents the piston sealing from moving out of the first cylindrical cavity, or
a mechanism which is configured to lock upon an insertion of the piston sealing into the first cylindrical cavity or which otherwise prevents the piston sealing from moving out of the first cylindrical cavity.

33. A method for operating the piston assembly as recited in claim 21, the method comprising:
providing the piston sealing which is arranged at the piston free from the sealing forces until use outside of the first cylindrical cavity or inside the first cylindrical cavity.

34. The method as recited in claim 33, wherein the piston is stored with the piston sealing in a relief cavity which is connected to the first cylindrical cavity.

35. A tribological system comprising:
two bodies which rub against each other in a contact range,
wherein,
at least one of the two bodies comprises, at least in the contact range, a thermoplastic elastomer or a thermoplastic material,
the thermoplastic elastomer or the thermoplastic material comprises a paraffin, and
the at least one of the two bodies is stored in air or in an oxygen comprising gas so that the paraffin moves to a surface of the at least one of the two bodies.

36. The tribological system as recited in claim 35, wherein the thermoplastic elastomer or the thermoplastic material is stored for at least six days.

37. The tribological system as recited in claim 36, wherein,
the at least one of the at least two bodies is a piston which comprises a piston sealing, and
the piston sealing comprises the thermoplastic elastomer which comprises the paraffin.

38. A method for reducing a friction in the tribological system as recited in claim 35, the method comprising:
providing a body which, at least in a contact range, comprises a thermoplastic elastomer or a thermoplastic material;
the thermoplastic elastomer or the thermoplastic material comprises a paraffin; and storing the body in air or in an oxygen containing gas before use so that the paraffin moves to a surface of the body.

39. The method as recited in claim 38, wherein,
the body is a piston which comprises a piston sealing, and
the piston sealing comprises the thermoplastic elastomer or the thermoplastic material comprising the paraffin.

40. The method as recited in claim 39, wherein,
the piston is guided in a cylindrical cavity having a diameter and an inner wall, and
the method further comprises:
providing a further body which has a diameter equal to or smaller than a diameter of the inner wall, the further body comprising a thermoplastic elastomer or a thermoplastic material which comprises a paraffin; and
coating the inner wall with the paraffin via the further body.
